(19) United States
(12) Patent Application Publication
(10) Pub. No.: US 2024/0042017 A1
(43) Pub. Date: Feb. 8, 2024

(54) METHODS FOR PREVENTING ORAL SENSITIZATION

(71) Applicant: MY-OR DIAGNOSTICS LTD., Zikhron Yaakov (IL)

(72) Inventors: Michael BRANDWEIN, Elazar (IL); Michal ASHKENAZI, Zurit (IL)

(21) Appl. No.: 18/275,481

(22) PCT Filed: Feb. 2, 2022
(86) PCT No.: PCT/IL2022/050143

§ 371 Date: Aug. 2, 2023

Related U.S. Application Data
(60) Provisional application No. 63/144,592, filed on Feb. 2, 2021.

(51) Int. Cl. A61K 39/35; A61K 45/06; A61K 9/00; A61P 37/08; A23L 33/00; A23L 33/19

(52) U.S. Cl. CPC A61K 39/35; A61K 45/06; A61K 9/0053; A61P 37/08; A23L 33/40; A23L 33/19

(57) ABSTRACT

The present invention relates a method for preventing sensitization to a food allergen in a subject in need thereof, including super early oral administration of a therapeutically effective amount of a composition comprising the food allergen to the subject, thereby preventing sensitization to the food allergen.

### CROSS-REFERENCE TO RELATED APPLICATIONS

This application claims the benefit of priority of U.S. Provisional Patent Application No. 63/144,592, titled “METHODS FOR PREVENTING ORAL SENSITIZATION” filed Feb. 2, 2021, the contents of which are incorporated herein by reference in their entirety.

### FIELD OF INVENTION

The present invention, in some embodiments thereof, is in the field of treating or preventing food sensitization.

### BACKGROUND

Allergic sensitization in childhood, especially in early childhood and especially food allergens, is critical and of the greatest interest since the development of an “allergic phenotype” or “atopy” has been shown to facilitate subsequent sensitization to other allergens. Therefore, childhood allergies may be the first stage of an allergic cascade that leads to multiple allergies later in life, a process commonly called “atopic gait.” For example, it has been shown in human cohorts that children with persistent food hypersensitivity at an early age have a dramatically increased risk of developing allergic rhinitis (hay fever) or asthma later in childhood. Children with milder forms of food hypersensitivity also have an increased risk of developing respiratory allergies but lesser degree than children with persistent food hypersensitivity. Therefore, attenuating the severity of food hypersensitivity can be crucial in slowing down the “atopic gait.” In this context, the control of allergic episodes and the prevention of allergies are, in childhood and childhood, of the utmost importance.

Food allergens are among the first allergens to which the infant is exposed at the beginning of his life. Typically, the infant not fed exclusively with breast milk is exposed to cow's milk proteins. Milk proteins are indeed among the most frequently observed causes of childhood food allergy, followed by eggs and wheat proteins. In general, food allergies can be manifested by skin symptoms (rashes, eczema, others) and gastrointestinal (abdominal spasms, pain, especially in the abdomen or vomiting) in infants and young children. Sensitization and additional allergy episodes may also occur when exposing the infant/toddler to a new food such as cereals, vegetables, fruits, nuts or fish.

There remains a need for methods for preventing sensitization to a food allergen in a subject in need thereof, such as, an infant.

### SUMMARY

According to a first aspect, there is provided a method for preventing sensitization to a food allergen in a subject in need thereof, the method comprising: (a) selecting a subject at a risk of developing sensitization to a food allergen; and (b) orally administering to the subject a therapeutically effective amount of a composition comprising the food allergen, wherein the administering commences when the subject being 28 days old at most, thereby preventing sensitization to a food allergen in the subject.

According to another aspect, there is provided a method for preventing sensitization to a food allergen in a subject in need thereof, the method comprising orally administering to the subject a therapeutically effective amount of a composition comprising the food allergen, wherein the composition is formulated in the form of drops, and wherein the administering commences when the subject being 28 days old at most, thereby preventing sensitization to a food allergen in the subject.

According to another aspect, there is provided a synthetic composition for use in preventing sensitization to a food allergen in a subject in need thereof, wherein the synthetic composition is in the form of drops, the synthetic composition comprises: (a) casein protein and whey protein in a weight per weight ratio (w/w) ranging from 55:45 (w/w) to 85:15 (w/w); and (b) a polyol at a volume of at least 40% by volume of the synthetic composition.

In some embodiments, the food allergen is selected from the group consisting of: non-human milk protein (hMP), egg protein, peanut, sesame, shellfish, fish, soy, grains, a tree nut, and any combination thereof.

In some embodiments, the sensitization comprises food protein-induced enterocolitis syndrome (FPIES), immunoglobulin E (IgE)-mediated, Type 2, or both.

In some embodiments, the preventing comprises reducing the severity of sensitization to the food allergen in the subject.

In some embodiments, the preventing comprises reducing the titer of IgE targeting the food allergen in the subject, reducing wheal size developed by the subject in a skin prick test (SPR), or both.

In some embodiments, the reducing comprises reducing the titer of IgE by at least 5% compared to a control subject.

In some embodiments, the reducing comprises reducing the wheal size to 1 mm at most.

In some embodiments, the administering is multiple administering.

In some embodiments, the multiple administering comprises daily administering.

In some embodiments, the multiple administering comprises at least once a week administering.

In some embodiments, the composition is a pharmaceutical composition or a nutraceutical composition.

In some embodiments, the composition further comprises an additional biomedical active agent.

In some embodiments, the additional biomedical active agent is selected from the group consisting of: a mineral, a vitamin, probiotics, and any combination thereof.

In some embodiments, the mineral comprises iron.

In some embodiments, the vitamin comprises vitamin D, vitamin B12, or any combination thereof.

In some embodiments, the composition is formulated in the form of drops.

In some embodiments, the food allergen is a non-hMP.

In some embodiments, the subject is fed strictly on human breast milk.

In some embodiments, the administering commences when the subject is 14 days old at most.

In some embodiments, the administering commences when the subject is 7 days old at most.

In some embodiments, the non-hMP comprises a cow milk protein (CMP).

In some embodiments, the non-hMP comprises cow milk formula (CMF).

In some embodiments, the casein comprises micellar casein.

In some embodiments, the polyol is glycerol.

In some embodiments, the synthetic composition is administered to a subject being 28 days old at most when administration commences.

In some embodiments, the synthetic composition comprises a total protein concentration ranging from 100 mg/ml to 350 mg/ml.

In some embodiments, the selecting is based on at least one criterion selected from the group consisting of: parent of said subject planning to exclusively breastfeed said subject, said subject having familial history of anyone of: atopic dermatitis, food allergy, allergic rhinitis, asthma, eosinophilic esophagitis, and any combination thereof, said subject being characterized by having an above-average/over a predetermined threshold of transepidermal water loss (TEWL) value, and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include vertical bar graphs showing the effect of early cow milk (CM) introduction on wheal size. (1A) A graph showing the effect of early CM introduction on “anytime skin prick test (SPT)>3”. (1B) A graph showing the effect of early CM introduction on Wheal size at each visit.

FIG. 2 includes a graph showing that the early introduction of peanuts does not influence the effects of early/late CM introduction presented in FIG. 1.

FIG. 3 includes scattered boxplots showing that Wheal size (e.g., disease severity) is significantly smaller in the early introduction group (CM formula (CMF)) when compared to the late introduction group. Wheal size was determined at the following time periods: 0, 12, 30, and 60 days. Note: Counting zeros.

FIGS. 4A-4B include vertical bar graphs showing the “tracking” of two groups of participants, grouped according to early introduction of CM (4A) vs late introduction of CM (4B), according to the Wheal presence (Y/N) at various times (0, 12, 30, and 60 days). Cutoff for Wheal—Yes is: Any Wheal>3=Yes.

FIGS. 5A-5M include micrographs showing different combinations of Casein and Why protein dissolved in glycerol solutions, and pasteurized at 72° C. for 20 sec. (5A) 0.6625 g total Casein, 80:20 Casein (Micellar casein): Whey, dissolved in 60% Glycerol, and 1.0% Sodium citrate; (5B) 0.6625 g total Casein, 80:20 Casein (Micellar casein): Whey, dissolved in 60% Glycerol; (5C) 5.000 g total Casein, 80:20 Casein (Micellar casein): Whey, dissolved in 60% Glycerol, and 1.0% Sodium citrate; (5D) 5.000 g total Casein, 80:20 Casein (Micellar casein): Whey, and dissolved in 60% Glycerol; (5E) 10.000 g total Casein, 80:20 Casein (Micellar casein): Whey, dissolved in 60% Glycerol, and 1.0% Sodium citrate; (5F) 10.000 g total Casein, 80:20 Casein (Micellar casein): Whey, and dissolved in 60% Glycerol; (5G) 0.6625 g total Casein, 60:40 Casein (Micellar casein): Whey, and dissolved in 60% Glycerol; (5H) 5.000 g total Casein, 60:40 Casein (Micellar casein): Whey, and dissolved in 60% Glycerol; (51) 10.000 g total Casein, 60:40 Casein (Micellar casein): Whey, and dissolved in 60% Glycerol; (5J) 0.6625 g total Casein, 80:20 Casein (Calcium casein): Whey, and dissolved in 50% Glycerol; (5K) 2.1625 g total Casein, 80:20 Casein (Calcium casein): Whey, and dissolved in 50% Glycerol; (5L) 3.6625 g total Casein, 80:20 Casein (Calcium casein): Whey, and dissolved in 50% Glycerol; and (5M) 5.000 g total Casein, 80:20 Casein (Calcium casein): Whey, and dissolved in 50% Glycerol.

### DETAILED DESCRIPTION

According to some embodiments, there is provided a method for preventing sensitization to a food allergen in a subject in need thereof.

In some embodiments, the method comprises: (a) selecting a subject at a risk of developing sensitization to a food allergen; and (b) orally administering to the subject a therapeutically effective amount of a composition comprising the food allergen, thereby preventing sensitization to a food allergen in the subject.

In some embodiments, the method comprises orally administering to the subject a therapeutically effective amount of a composition comprising the food allergen, wherein the composition is formulated in the form of drops.

In some embodiments, a food allergen comprises any food or a compound derived therefrom that induces an abnormal immune response when orally consumed by a subject. In some embodiments, a food allergen comprises or is a protein or a plurality of proteins derived from a food product. In some embodiments, an abnormal immune response comprises production of an immunoglobulin in a response to the consumption of a food product. In some embodiments, the immunoglobulin is immunoglobulin E (IgE).

In some embodiments, the administering commences when the subject is at least 1 day old, at least 2 days old, at least 3 days old, at least 4 days old, at least 5 days old, at least 7 days old, at least 9 days old, at least 10 days old, at least 12 days old, at least 14 days old, at least 16 days old, at least 19 days old, at least 21 days old, at least 25 days old, at least 28 days old, or at least 30 days old, or any value ad range therebetween. Each possibility represents a separate embodiment of the invention.

In some embodiments, the administering commences when the subject is 1 day old at most, 2 days old at most, 3 days old at most, 4 days old at most, 5 days old at most, 7 days old at most, 9 days old at most, 10 days old at most, 12 days old at most, 14 days old at most, 16 days old at most, 19 days old at most, 21 days old at most, 25 days old at most, 28 days old at most, or 30 days old at most, or any value ad range therebetween. Each possibility represents a separate embodiment of the invention.

In some embodiments, the administering commences when the subject is 1 day to 14 days, 2 days to 30 days, 1 day to 10 days old, 4 days to 16 days old, 5 days to 15 days old, 1 day to 7 days, or 3 days to 18 days Each possibility represents a separate embodiment of the invention.

In one embodiment, a subject as described herein is an infant. In one embodiment, the infant is 2 weeks old at most. In another embodiment, the infant is approximately 3 months old. In another embodiment, the subject is a child.

In some embodiments, the administering commences when the subject is 28 days old at most.

In some embodiments, the food allergen is selected from: non-human milk protein (hMP), egg protein, peanut, sesame, shellfish, fish, soy, grains, tree nut, or any combination thereof.

In some embodiments, the food allergen comprises a peptide, a protein, or any plurality thereof, being derived from: non-human milk protein (hMP), egg protein, peanut, sesame, shellfish, fish, soy, grains, tree nut, or any combination thereof.

In some embodiments, sensitization comprises food protein-induced enterocolitis syndrome (FPIES), immunoglobulin E (IgE)-mediated, Type 2.

It is to be stressed out here, that sensitization alone is not sufficient to diagnose food allergy, nor is synonymous or equivalent thereto.

In some embodiments, sensitization and allergy are not synonymous or equivalent.

In some embodiments, sensitization is characterized by: the development of specific IgE (sIgE) capable of targeting and/or binding to a food allergen, such as, but not limited to a non-hMP by a subject, lack of clinical symptoms after the subject has been exposed to a food allergen, such as a non-hMP, or both.

In some embodiments, allergy to a food allergen is characterized by the development of clinical symptoms after a subject has been exposed to the food allergen.

Methods for determining IgE titer, clinical symptoms, or both, are common and would be apparent to one of ordinary skill in the art of medicine.

In some embodiments, a subject in need of prevention according to the herein disclosed method is at risk of developing sensitization to a food allergen. In some embodiments, the method disclosed herein is directed to prevent sensitization to a food allergen. In some embodiments, the method of the invention is not directed to the treatment of food allergy, wherein the allergen is or comprises a food allergen.

In some embodiments, the preventing comprises reducing the severity of sensitization to the food allergen in the subject.

In some embodiments, the method comprises reducing the severity of sensitization to a food allergen in the subject.

In some embodiments, preventing comprises reducing the titer of immunoglobulin E (IgE) targeting a food allergen (such as, but not limited to a CMP) in the subject, reducing wheal size developed by the subject in a skin prick test (SPT), or both.

In some embodiments, the method comprises reducing a wheal size to 0.1 mm at most, 0.3 mm at most, 0.5 mm at most, 0.75 mm at most, 1 mm at most, or any value and range therebetween. Each possibility represents a separate embodiment of the invention. In some embodiments, the method comprises reducing a wheal size to 0.1-0.8 mm, 0.2-1.0 mm, 0.3-0.75 mm, or 0.2-0.9 mm. Each possibility represents a separate embodiment of the invention.

In some embodiments, reducing is by at least 5%, at least 15%, at least 25%, at least 50%, at least 100%, at least 200%, at least 350%, at least 500%, at least 750%, at least 1,000%, or any value and range therebetween. Each possibility represents a separate embodiment of the invention. In some embodiments, reducing is by 5-200%, 5-50%, 100-250%, 200-750%, 10-350%, 90-450%, or 150-1,000%. Each possibility represents a separate embodiment of the invention. In some embodiments, reducing is compared to a control subject, as described herein.

In reducing comprises reducing the titer of IgE by at least 5% compared to a control subject.

As used herein, the term “prevention” refers to reducing the susceptibility, delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, preventing comprises reducing the susceptibility of a subject administered with the composition according to the method of the invention, to develop oral sensitization to a food allergen. In some embodiments, a subject administered with the composition according to the method of the invention, has reduced susceptibility of developing oral sensitization to a food allergen, compared to a control subject. In some embodiments, a control subject is not administered with the composition according to the method of the invention.

As used herein, the term “at risk” refers to the susceptibility of a subject to developing a condition, such as sensitization to a food allergen. In some embodiments, detecting or determining a risk comprises detecting the presence of the disease itself. In some embodiments, detecting or determining a risk comprises any one of: determining the susceptibility of the subject to developing the condition, having a poor prognosis for the condition, having increased severity of the condition, or any combination thereof.

In some embodiments, “at risk” refers to increased probability of developing, or having increased predisposition, compared to a control. In some embodiments, the control comprises the average population.

The terms “infant”, “baby”, “preemie”, “newborn”, are used herein interchangeably.

In some embodiments, preventing a sensitization to a food allergen (e.g., “the condition”) comprises reducing the condition severity, delaying the condition onset, reducing the condition cumulative incidence, or any combination thereof.

In some embodiments, administering is multiple administering.

In some embodiments, the method comprises multiple administering. In some embodiments, the method comprises daily administering. In some embodiments, multiple administering comprises daily administering. In some embodiments, multiple administering comprises at least once a week, at least twice a week, at least once every other week, at least once in two weeks, at least once in three weeks, at least once a month administering, or any value and range therebetween. Each possibility represents a separate embodiment of the invention. In some embodiments, multiple administering comprises once a week to once a month administering.

In some embodiments, the composition is a pharmaceutical composition or a nutraceutical composition.

According to another aspect, there is provided a composition for use in preventing sensitization to a food allergen in a subject in need thereof, wherein the composition is in the form of drops.

In some embodiments, the composition comprises: (a) casein protein and whey protein in a weight per weight ratio (w/w) ranging from 55:45 (w/w) to 85:15 (w/w); and (b) a polyol at a volume of at least 40% by volume of the synthetic composition.

In some embodiments, the composition is a synthetic composition.

As used herein, the term “synthetic” refers to being manmade.

The term “synthetic” is interchangeable herein with the term “artificial”.

In some embodiments, the polyol comprises at least 40%, at least 50%, at least 60%, at least 70%, at least 80%, at least 90%, at least 95%, or at least 99% by volume of the composition, or any value and range therebetween. Each possibility represents a separate embodiment of the invention. In some embodiments, the polyol comprises 40-60%, 45-80%, 50-90%, or 55-99% by volume of the composition. Each possibility represents a separate embodiment of the invention.

Types of polyol would be apparent to one of ordinary skill in the art.

In some embodiments, the polyol comprises a plurality of types of polyols.

As used herein, the term “plurality” refers to any integer equal to or greater than 2.

In some embodiments, the polyol or a plurality there comprises or consists of glycerol (e.g., glycerin).

In some embodiments, the polyol is dissolved or mixed in a solvent. In some embodiments, the solvent comprises a polar solvent. In some embodiments, the solvent is or comprises water.

In some embodiments, the composition comprises water. In some embodiments, the composition comprises glycerol. In some embodiments, the composition comprises glycerol dissolved or mixed with water. In some embodiments, the composition comprises a solution comprising 30-60%, 50-70%, or 35-85% (v/v) glycerol in water.

In some embodiments, the composition comprises casein and whey protein at a weight per weight ratio (w/w) of about 50:50 (w/w)±10%, 55:45 (w/w)+10%, 60:40 (w/w)±10%, 65:35 (w/w)±10%, 70:30 (w/w)±10%, 75:35 (w/w)±10%, 80:20 (w/w)±10%, 85:15 (w/w)+10%, or 90:10 (w/w)+10%. Each possibility represents a separate embodiment of the invention.

In some embodiments, the composition comprises casein and whey protein at a weight per weight ratio (w/w) ranging from 50:50 (w/w) to 90:10 (w/w), 60:40 (w/w) to 90:10 (w/w), 70:30 (w/w) to 90:10 (w/w), or 80:20 (w/w) to 90:10 (w/w). Each possibility represents a separate embodiment of the invention.

In some embodiments, the composition comprises casein and whey protein at a weight per weight ratio (w/w) ranging from 60:40 (w/w) to 80:20 (w/w).

In some embodiments, the composition comprises a total protein amount of at least 10 mg/ml, at least 20 mg/ml, at least 50 mg/ml, at least 75 mg/ml, at least 100 mg/ml, at least 150 mg/ml, at least 200 mg/ml, at least 300 mg/ml, at least 500 mg/ml, or at least 1,000 mg/ml, or any value and range therebetween. Each possibility represents a separate embodiment of the invention. In some embodiments, the composition comprises a total protein amount of 10-100 mg/ml, 50-500 mg/ml, 100-600 mg/ml, or 100-300 mg/ml. Each possibility represents a separate embodiment of the invention.

In some embodiments, the composition is administered to a subject being 28 days old at most when administration commences.

In some embodiments, the composition further comprises a pH regulating agent.

In some embodiments, the pH regulating agent is regulating acidity levels.

In some embodiments, the pH regulating agent comprises or is sodium citrate.

In some embodiments, the composition further comprises sodium citrate.

In some embodiments, the composition comprises sodium citate in an amount of at least 0.001%, at least 0.01%, at least 0.1%, at least 0.5%, at least 1.00%, at least 2.00%, or at least 2.50% by weight of the composition, or any value and range therebetween. Each possibility represents a separate embodiment of the invention.

In some embodiments, the composition comprises sodium citate in an amount of 0.00% to 1.5% by weight of the composition.

In some embodiments, casein comprises or is micellar casein.

The term “micellar casein” would be apparent to one of ordinary skill in the art, such as described in any one of WO2016/174651, and WO2017/003708.

According to some embodiments, there is provided a method for preparing or producing the composition disclosed herein.

In some embodiments, the method comprises pasteurizing the composition.

In some embodiments, the method comprises sterilizing the composition.

In some embodiments, pasteurizing comprises sterilizing.

In some embodiments, pasteurizing comprises heating or subjecting to a temperature of 60-80° C., 65-80° C., 65-75° C., 68-75° C., or 70-74° C. Each possibility represents a separate embodiment of the invention.

In some embodiments, pasteurizing is for a period of at least 2 sec, at least 5 sec, at least 10 sec, at least 15 sec, at least 20 sec, at least 25 sec, at least 30 sec, at least 45 sec, or at least 60 sec, or any value and range therebetween. Each possibility represents a separate embodiment of the invention. In some embodiments, pasteurizing is for a period of 1-60 sec, 2-40 sec, 3-30 sec, or 5-25 sec. Each possibility represents a separate embodiment of the invention.

In some embodiments, pasteurizing comprises heating or subjecting to a temperature of 60-80° C., 65-80° C., 65-75° C., 68-75° C., or 70-74° C. for a period of 1-60 sec, 2-40 sec, 3-30 sec, or 5-25 sec. Each possibility represents a separate embodiment of the invention.

In some embodiments, the composition further comprises an additional biomedical active agent.

In some embodiments, the additional biomedical active agent is selected: a mineral, a vitamin, probiotics, or any combination thereof.

In some embodiments, the mineral comprises iron.

In some embodiments, the vitamin comprises vitamin D, vitamin B12, or any combination thereof.

In some embodiments, the composition is formulated in the form of drops. In some embodiments, the composition is formulated in any way known to one of skill in the art as suitable for administration in a baby, an infant, or a newborn.

In some embodiments, the composition is administered in conjugation to breast feeding.

In some embodiments, the subject is fed strictly on human breast milk. In some embodiments, the subject is exclusively breastfed.

In some embodiments, the food allergen is a non-hMP.

In one embodiment, a non-hMP comprises milk protein derived or obtained from any habited organism, such as, but not limited to, cow, goat, sheep, camel, buffalo, and others.

In some embodiments, a non-hMP comprises or consists of a cow milk protein (CMP).

In some embodiments, the non-hMP comprises cow milk formula (CMF). In some embodiments, CMP comprises cow milk formula (CMF).

As used herein, the term “CMF” refers to any human breast milk replacement produced artificially and/or synthetically by man. Types of CMF are common and would be apparent to one of ordinary skill in the art.

According to some embodiments, there is provided a method for preventing sensitization to a non-human milk protein (hMP) in a subject in need thereof, wherein the subject is an infant strictly or exclusively fed on human breast milk.

In some embodiments, the herein disclosed method is directed to providing oral tolerance of a subject to a non-hMP. In some embodiments, the composition disclosed herein is provided to induce oral tolerance of a subject to a non-hMP. In some embodiments, the herein disclosed method and/or composition are directed to provided oral tolerance of a subject to a non-hMP and not as a replacement to human MP and/or breast feeding. In some embodiments, replacement comprises partial or full replacement.

In some embodiments, the subject is fed on a diet consisting of human breast milk. In some embodiments, the subject is being breast fed. In some embodiments, the subject is fed on pumped human breast milk.

In some embodiments, selecting is based on at least one criterion selected from: parent of the subject planning to exclusively breastfeed the subject, the subject having familial history of anyone of: atopic dermatitis, food allergy, allergic rhinitis, asthma, eosinophilic esophagitis, or any combination thereof, the subject being characterized by having an above average/over a predetermined threshold of transepidermal water loss (TEWL) value, or any combination thereof.

Methods for determining TEWL are common and would be apparent to one of skill in the art, as well as what is considered normal, proper, adequate, or average, TEWL value.

Effective doses of the compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, also will depend on the nature of the disorder or condition and can be determined by standard clinical techniques.

For therapeutic purposes, the active compounds of this invention are ordinarily combined with one or more adjuvants appropriate to the indicated route of administration.

Compositions of the invention optionally comprise an additional agent selected from any pharmaceutically or nutraceutically acceptable carrier, adjuvant, and vehicle.

### GENERAL

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise,” or variations such as “comprises” or “comprising,” indicate the inclusion of any recited integer or group of integers but not the exclusion of any other integer or group of integers.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Example 1

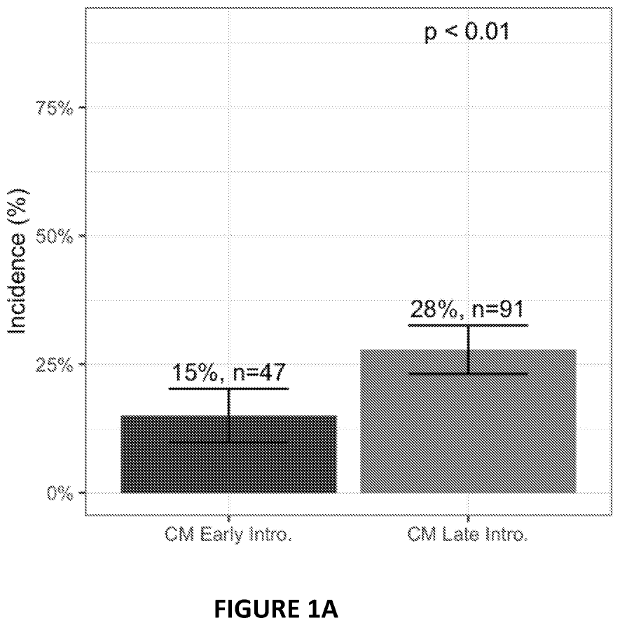

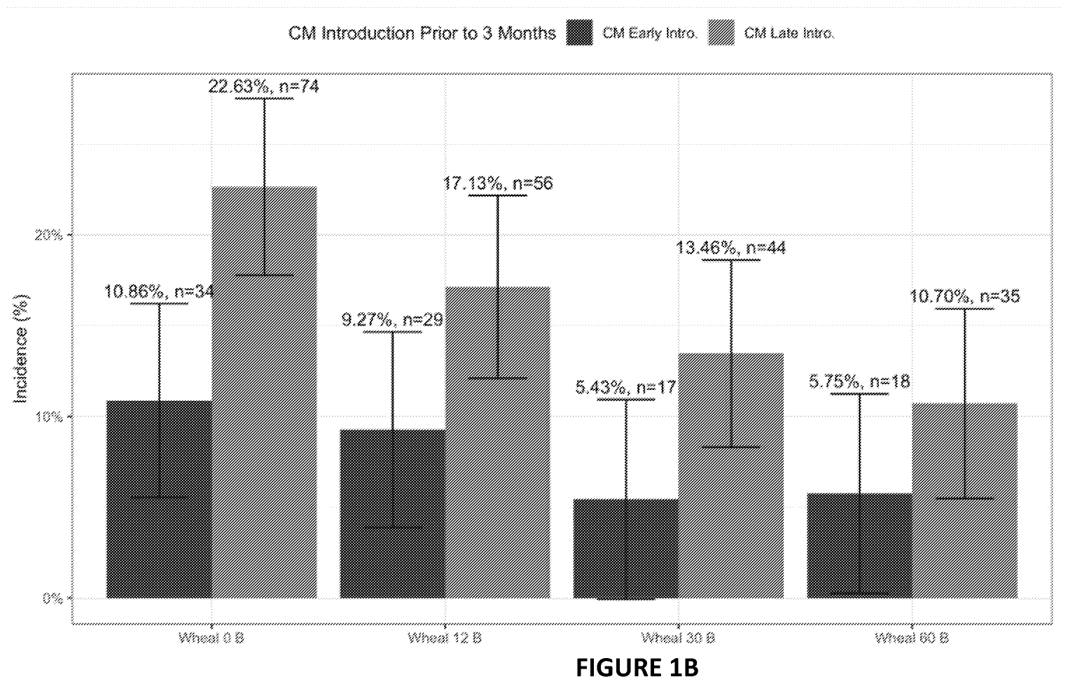

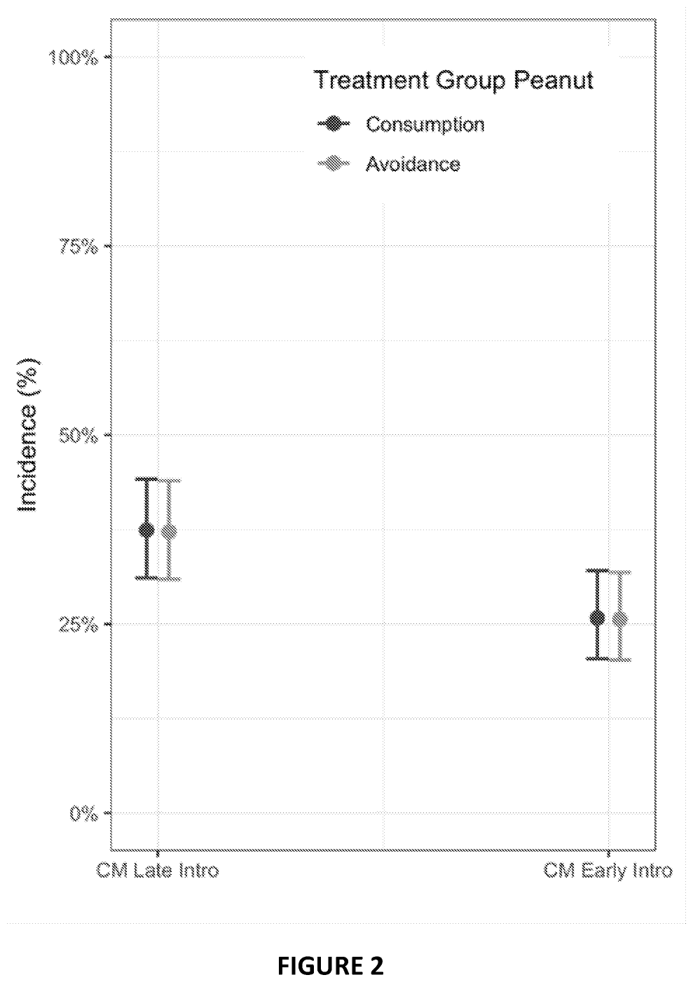

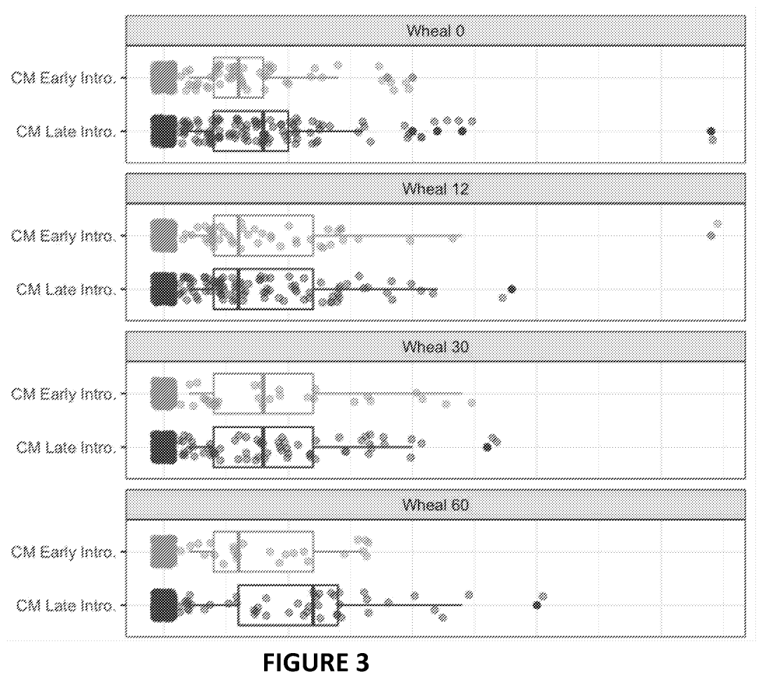

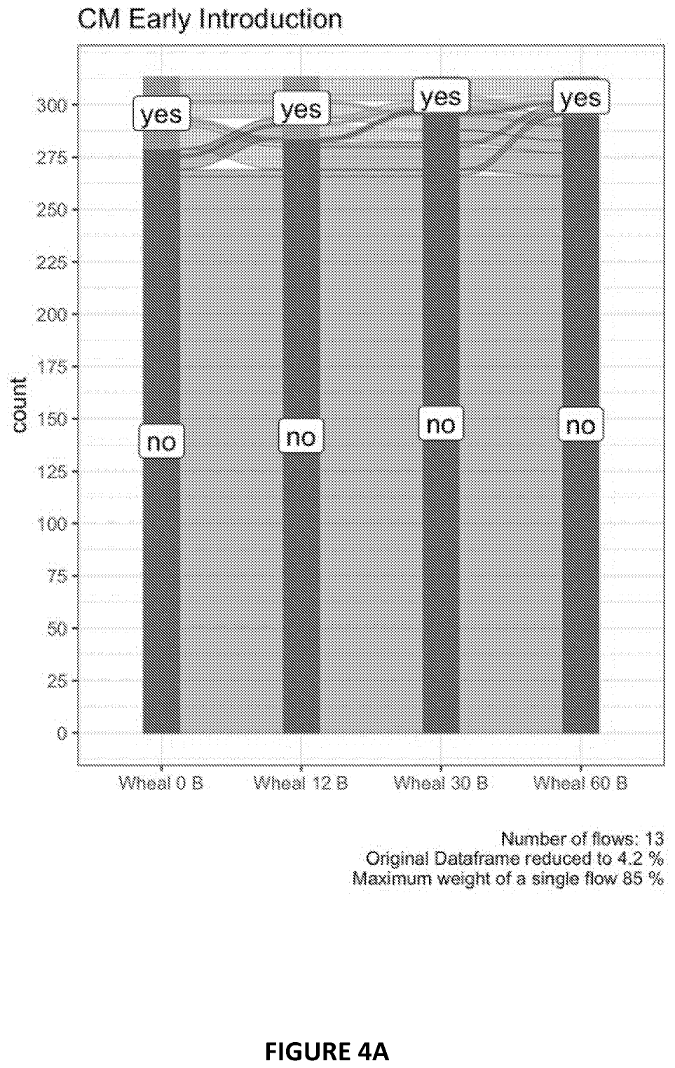

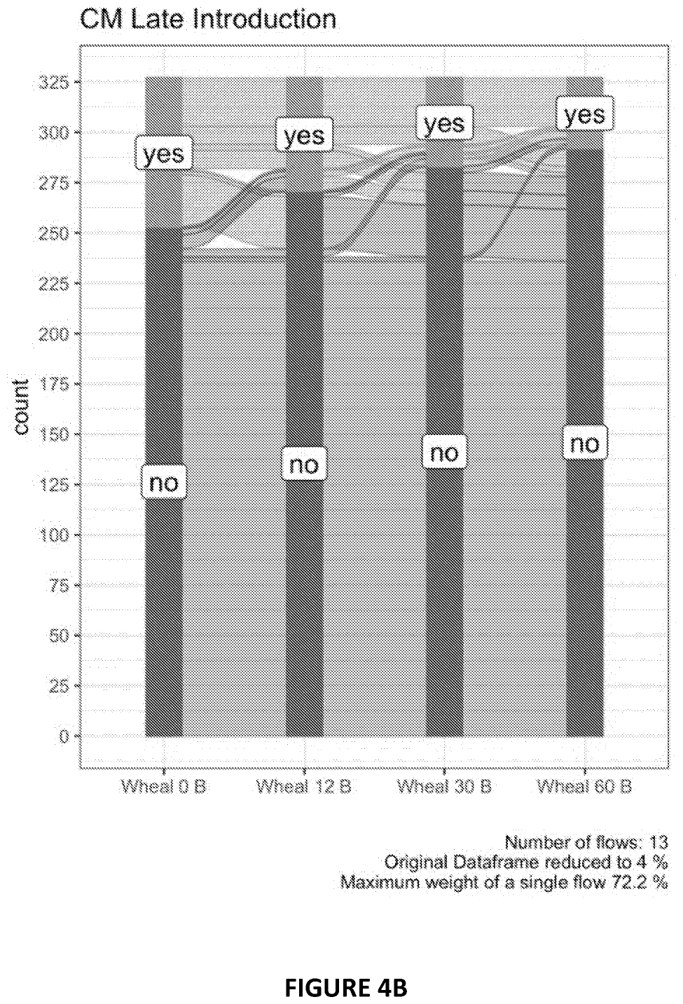

Infants were recruited at age 4-11 months and provided questionnaires to assess the timing of cow's milk formula, the cessation of exclusive breastfeeding and other feeding practices. Skin prick tests were carried out at recruitment, 12 months, 30 months and 60 months. Cow's milk IgE was measured at baseline. Sensitivity was defined as a wheal size of greater than 3 mm for SPT or CM IgE levels greater than 0.35. Analysis indicated that the introduction of allergens prior to 3 months of age resulted in a significant prevention of sensitivity in ˜50% of the population at baseline and all subsequent visits (FIG. 1B).

### Example 2

### Formulation of Casein/Whey Protein Drops

The inventors have examined different combinations of casein and whey protein so as to obtain a formulation comprising casein and whey protein that is suitable to be used as drops according to the herein disclosed method.

The following formulations were prepared. Thereafter, the inventors have examined solution coloration, protein precipitation, turbidity, based on which, suitable specifications, were determined.

In general, all formulations were prepared such that the final protein concentration was 150 mg/ml.

TABLE 1
Formulations specification and analysis.

| Evaluation | Comments | FIG. |
|---|---|---|
| 0.6625 g total casein 80:20 | Heat was applied to disperse casein, | 5A |
| Casein*:Whey, 60% Glycerol, | plus, to reduce microbial load. After 1 | |
| 1.0% Sodium citrate. | month, no apparent change in | |
| Pasteurization 72° C. for 20 sec. | turbidity. A thin dispersed layer of | |
| *Micellar casein | protein precipitated. | |
| 0.6625 g total casein 80:20 | Heat was applied to disperse casein, | 5B |
| Casein*:Whey, 60% Glycerol, | plus, to reduce microbial load. After 1 | |
| 0.0% Sodium citrate. | month, no apparent change in | |
| Pasteurization 72° C. for 20 sec. | turbidity. A concentrated layer of | |
| *Micellar casein | protein precipitated. Color of micellar | |
| | casein changed from white to brown. | |
| 5.000 g total casein 80:20 | Heat was applied to disperse casein, | 5C |
| Casein*:Whey, 60% Glycerol, | plus, to reduce microbial load. After 1 | |
| 1.0% Sodium citrate. | month, there was a change in | |
| Pasteurization 72° C. for 20 sec. | turbidity. A dispersed layer of protein | |
| *Micellar casein | precipitated. Color of micellar casein | |
| | changed from white to brown. | |
| 5.000 g total casein 80:20 | Heat was applied to disperse casein, | 5D |
| Casein*:Whey, 60% Glycerol, | plus, to reduce microbial load. After 1 | |
| 0.0% Sodium citrate. | month, there was a change in | |
| Pasteurization 72° C. for 20 sec. | turbidity. A dispersed layer of protein | |
| *Micellar casein | precipitated. Color of micellar casein | |
| | changed from white to brown. | |
| 10.000 g total casein 80:20 | Heat was applied to disperse casein, | 5E |
| Casein*:Whey, 60% Glycerol, | plus, to reduce microbial load. After 1 | |
| 1.0% Sodium citrate. | month, there was a change in | |
| Pasteurization 72° C. for 20 sec. | turbidity. A dispersed layer of protein | |
| *Micellar casein | precipitated. Color of micellar casein | |
| | changed from white to brown. | |
| 10.000 g total casein 80:20 | Heat was applied to disperse casein, | 5F |
| Casein*:Whey, 60% Glycerol, | plus, to reduce microbial load. After a | |
| 0.0% Sodium citrate. | month, there was a change in | |
| Pasteurization 72° C. for 20 sec. | turbidity. A layer of protein | |
| *Micellar casein | precipitated. Color of micellar casein | |
| | changed from white to brownish. | |
| 0.6625 g total casein 60:40 | Heat was applied to disperse casein, | 5G |
| Casein*:Whey, 60% Glycerol. | plus, to reduce microbial load. After a | |
| Pasteurization 72° C. for 20 sec. | month, there was a change in | |
| *Micellar casein | turbidity. A layer of protein | |
| | precipitated. | |
| 5.000 g total casein 60:40 | Heat was applied to disperse casein, | 5H |
| Casein*:Whey, 60% Glycerol. | plus, to reduce microbial load. After a | |
| Pasteurization 72° C. for 20 sec. | week, there was a change in turbidity. | |
| *Micellar casein | A layer of protein precipitated. Color | |
| | of micellar casein changed from white | |
| | to brownish. | |
| 10.000 g total casein 60:40 | Heat was applied to disperse casein, | 5I |
| Casein*:Whey, 60% Glycerol. | plus, to reduce microbial load. After a | |
| Pasteurization 72° C. for 20 sec. | week, there was a change in turbidity. | |
| *Micellar casein | A layer of protein precipitated. Color | |
| | of micellar casein changed from white | |
| | to brownish. | |
| 0.6625 g total casein 80:20 | Heat was applied to disperse casein, | 5J |
| Casein*:Whey, 50% Glycerol. | plus, to reduce microbial load. After a | |
| Pasteurization 72° C. for 20 sec. | week, there was a change in turbidity. | |
| *Calcium casein | A layer of protein precipitated. | |
| 2.1625 g total casein 80:20 | Heat was applied to disperse casein, | 5K |
| Casein*:Whey, 50% Glycerol. | plus, to reduce microbial load. After a | |
| Pasteurization 72° C. for 20 sec. | week, there was a change in turbidity. | |
| *Calcium casein | A layer of protein precipitated. | |
| 3.6625 g total casein 80:20 | Heat was applied to disperse casein, | 5L |
| Casein*:Whey, 50% Glycerol. | plus, to reduce microbial load. After a | |
| Pasteurization 72° C. for 20 sec. | week, there was a change in turbidity. | |
| *Calcium casein | A layer of protein precipitated. | |
| 5.000 g total casein 80:20 | Heat was applied to disperse casein, | 5M |
| Casein*:Whey, 50% Glycerol. | plus, to reduce microbial load. After a | |
| Pasteurization 72° C. for 20 sec. | week, there was a change in turbidity. | |
| *Calcium casein | A layer of protein precipitated. | |

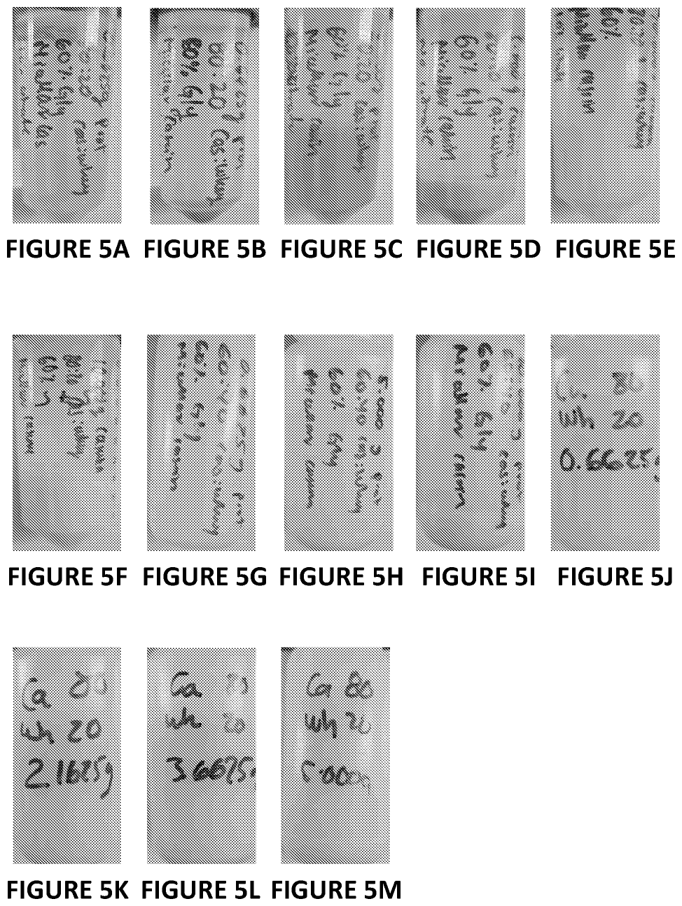

The inventors found the formulations of FIGS. 5A and 5G to be satisfactory.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for preventing sensitization to a food allergen in a subject in need thereof, the method comprising:
a. selecting a subject at a risk of developing sensitization to a food allergen; and
b. orally administering to said subject a therapeutically effective amount of a composition comprising said food allergen, wherein said administering commences when said subject being 28 days old at most,
thereby preventing sensitization to a food allergen in the subject.

2. The method of claim 1, wherein said composition is formulated in the form of drops, and optionally wherein said selecting is based on at least one criterion selected from the group consisting of: parent of said subject planning to exclusively breastfeed said subject, said subject having familial history of anyone of: atopic dermatitis, food allergy, allergic rhinitis, asthma, eosinophilic esophagitis, and any combination thereof, said subject being characterized by having an above-average/over a predetermined threshold of transepidermal water loss (TEWL) value, and any combination thereof.

3. (canceled)

4. A method for preventing sensitization to a food allergen in a subject in need thereof, the method comprising orally administering to said subject a therapeutically effective amount of a composition comprising said food allergen, wherein said composition is formulated in the form of drops, and wherein said administering commences when said subject being 28 days old at most, thereby preventing sensitization to a food allergen in the subject.

5. The method of claim 1, wherein said food allergen is selected from the group consisting of: non-human milk protein (hMP), egg protein, peanut, sesame, shellfish, fish, soy, grains, a tree nut, and any combination thereof, and optionally wherein any one of: said non-hMP comprises a cow milk protein (CMP), and said non-hMP comprises cow milk formula (CMF).

6. The method of claim 1, wherein said sensitization comprises food protein-induced enterocolitis syndrome (FPIES), immunoglobulin E (IgE)-mediated, Type 2, or both.

7. The method of claim 1, wherein said preventing comprises reducing the severity of sensitization to said food allergen in the subject.

8. The method of claim 1, wherein said preventing comprises reducing the titer of IgE targeting said food allergen in said subject, reducing wheal size developed by said subject in a skin prick test (SPR), or both.

9. The method of claim 8, wherein said reducing comprises reducing said titer of IgE by at least 5% compared to a control subject.

10. The method of claim 8, wherein said reducing comprises reducing said wheal size to 1 mm at most.

11. The method of claim 1, wherein said administering is multiple administering.

12. The method of claim 11, wherein said multiple administering comprises daily administering.

13. The method of claim 11, wherein said multiple administering comprises at least once a week administering.

14. The method of claim 1, wherein said composition is a pharmaceutical composition or a nutraceutical composition.

15. The method of claim 1, wherein said composition further comprises an additional biomedical active agent.

16. The method of claim 15, wherein said additional biomedical active agent is selected from the group consisting of: a mineral, a vitamin, probiotics, and any combination thereof, and optionally wherein said mineral comprises iron.

17. (canceled)

18. The method of claim 16, wherein said vitamin comprises vitamin D, vitamin B12, or any combination thereof.

19. The method of claim 1, wherein said food allergen is a non-hMP.

20. The method of claim 1, wherein said subject is fed strictly on human breast milk.

21. The method of claim 19, wherein said administering commences when said subject being 14 days old at most.

22. The method of claim 19, wherein said administering commences when said subject being 7 days old at most.

23.-29. (canceled)
